(19) United States
(12) Patent Application Publication
(10) Pub. No.: US 2013/0309291 A1
(43) Pub. Date: Nov. 21, 2013

(54) CONFECTION COMPOSITION

(71) Applicant: Andrea Stoll, Andover, MA (US)

(72) Inventor: Andrea Stoll, Andover, MA (US)

(73) Assignee: SNAP INFUSION LLC, Andover, MA (US)

(21) Appl. No.: 13/982,550

(22) PCT Filed: Feb. 3, 2012
(86) PCT No.: PCT/US2012/023743

§ 371 Date: Aug. 2, 2013

Related U.S. Application Data
(60) Provisional application No. 61/462,464, filed on Feb. 3, 2011.

(51) Int. Cl. A23G 3/36; A23G 3/54; A23G 4/20; A23G 4/06; A61K 36/185; A61K 9/00; A61K 31/352; A61K 38/48; A61K 31/198; A61K 36/64; A23G 3/44; A23G 1/32

(52) U.S. Cl. CPC A23G 3/368; A23G 3/36; A23G 3/364; A23G 3/44; A23G 3/54; A23G 4/20; A23G 4/06; A23G 1/32; A61K 9/0056; A61K 31/352; A61K 38/48; A61K 31/198; A61K 36/64; A61K 36/185
USPC 424/440; 426/660; 426/74; 426/73; 426/72; 426/648; 426/655; 426/656; 426/103; 426/5; 426/3; 424/184.1

(57) ABSTRACT

The present embodiments provide for edible confectionery compositions made of natural ingredients, comprising a confectionery main body that includes, within the main body, a confectionery functional ingredient vehicle. The functional ingredient vehicle can include, for example, vitamins, antioxidants and/or electrolytes. The vehicle also provides at least one sensory signal to the consumer, such as a snap, crunch or pop, characterized by a hard bite-through.

### RELATED APPLICATIONS

The present application claims priority benefit of U.S. Ser. No. 61/462,464, filed 3 Feb. 2011, and incorporated fully herein by this reference.

### FIELD

The present embodiments relate to edible, natural ingredient-based confectionery compositions comprising functional ingredients, such as vitamins, antioxidants and/or electrolytes, in conjunction with a sensory indicator vehicle that provides a palpable indicator, a visual indicator, or an audible indicator.

### BACKGROUND

Confections such as candies, sweets, and chewing gum are typically sugar-based snacks with little or no nutritional value. Even sugar-free versions of these confections rarely confer any nutritional value. Not generally associated with meals or prescribed therapies, candies and chewing gum are often considered guilty pleasures. Additionally, many current confections include artificial colors, flavors and/or sweeteners, further subtracting from the nutritional value of such snacks. Nevertheless, globally, we spend over $100 billion on candy every year, and overall candy consumption continues to increase. Americans alone consume, on average, 5.4 kg of sugar candy and 6.5 kg of chocolate per person annually, about 65% of which is consumed by adults. Additionally, the average person chews over 300 sticks of gum each year. Given that humans consume these products for pleasure, there is a need for confections that provide for an enhanced pleasurable, i.e., sensory, experience that is combined with functional ingredients for enhanced nutritional value and eating pleasure.

### SUMMARY

The present embodiments provide for confectionery snacks with enhanced sensory and nutritional value. The confectionery snack, which is natural ingredient-based, can be a hard candy, soft candy, chewing gum, caramel, jelly bean, extruded food stuff, pressed tablet, coated or uncoated confection, or any other known confection type. The present embodiments provide for a natural ingredient-based confection that contains at least one functional ingredient (e.g., a vitamin, antioxidant, electrolyte or other beneficial agent) in or with a vehicle that also provides palpable, visual, or audible signal to the consumer. More specifically, the palpable or audible indicator is a snap, crunch, or pop associated with a hard bite-through of at least one encapsulation within the confection. Particular embodiments provide for improved confections comprising effective amounts of shelf-stable antioxidants, that also provides a sensory (e.g., visual, tactile, or audible) indicator to the consumer.

In particular embodiments, the confection includes at least one functional ingredient comprising a water-insoluble or lipophilic (fat-soluble) dietary supplement in an encapsulated medium with a standardized shell hardness and optional natural colorant. Thus, for example, a serving size of the confection (e.g., one piece of gum or a single-serving package of many candies) can include (percentage daily values based on a 2,000 calorie diet) about 2% to about 45%, inclusive, of at least one lipophilic vitamin, such as vitamin E.

In the embodiments of the invention, the visual sensory indicators (i.e., color and/or shape) and mouthfeel sensory indicators (e.g., snap, tang, crunch) and, optionally, audible indicators, (i.e., crunch, snap, pop) are particularly advantageous to identify these confections as having more nutritional value than typical snacks consumed for pleasure, and such indicators may deliver information regarding the functional ingredient. The hardness of the encapsulation can be selected to provide for a hard bite-through, and/or an audible crunch upon mastication, further enforcing the consumer's knowledge of the nutritional value of the confection while adding to the fun of eating the confection. The size of the functional ingredient vehicle has an average diameter selected from 5 μm to 800 μm, such as 50 μm to 300 μm. The color and shape of the encapsulation can also be chosen depending upon what type of medicament is contained. For example, orange-colored spherical encapsulations may comprise the supplement ascorbyl palmitate, while red-colored heart-shaped encapsulations may comprise essential fatty acids, such that the user gains dietary information as well as pleasure upon observation of the confection. Thus, in some embodiments, the confection composition contains an encapsulated medicament in a vehicle that acts a visual and palpable/audible signal to the consumer.

The encapsulated compositions can further comprise both water-soluble and water-insoluble ingredients. The water-insoluble ingredients can be included in the confection where appropriate or may be complexed onto the shell of an encapsulated medicament. The encapsulated medicament may be included into any known confection type with various levels of moisture content.

In other embodiments, the encapsulated functional ingredient is one that is sensitive to light, heat, water, oxidation or is incompatible with ingredients in the confection composition. In other embodiments, the functional ingredient is a homeopathic remedy, such as an herb or spice associated with homeopathic treatment.

In some embodiments, the encapsulation can have a size ranging from 0.01 mm to 30 mm, inclusive, depending upon the application (e.g., candy type or gum) and functional ingredient. The shell hardness and thickness can be determined depending upon the functional ingredient and the confection type (e.g., candy type or gum) in which it is included. The hardness and thickness can be formulated to enhance the mouthfeel, e.g., a palpable snap or crunch, of the confection. Additionally, the hardness and thickness can be formulated to enhance audible indicator (i.e., sound) of the confection upon mastication. The shell of the encapsulation can have various types of coatings and combinations, and/or layers including pectin, gelatin, sugar, sugar free, hard, semi-hard, and soft coating by various coating techniques to further increase the audible signal when chewed, or to further protect the medicament from oxidation or rancidity.

The resulting confectioneries help consumers identify confections that contain functional ingredients visually, and palpably and/or audibly when chewed, while protecting the enclosed medicaments from oxidation and increasing the shelf-life of the medicaments that are encapsulated. The confection composition containing these encapsulated medicaments are also more palatable to the consumer, and thus result in increased consumption of the confections on a daily basis, which is beneficial for daily vitamin requirements.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present embodiments provide for confectioneries (confections, candy, chewing gum, etc.) with improved consumption experience. More specifically, the present embodiments provide for a natural ingredient-based confectionery having increased nutritional benefit, comprising functional agents, associated with sensory indicators, such as visual, audible, and mouthfeel indicators. For example, vitamins and/or antioxidants are encapsulated in a manner that provides a pleasing crunch and/or snap sound upon mastication, and the encapsulated functional ingredient is colored to provide pleasing or informative colors to the confectionery.

Examples of confectionery, as used herein, include a variety of candies and chewing gums. Candy varieties are typically influenced by the size of the sugar crystals, aeration, sugar concentrations, color and the types of sugar or natural sugar substitute used. Hard sweets are based on sugars cooked to the hard-crack stage: Examples include suckers (known as boiled sweets in British English), lollipops, jawbreakers (or gobstoppers), lemon drops, peppermint drops and disks, candy canes, rock candy, etc. These also include types often mixed with nuts such as brittle. Others contain flavorings including coffee such as Kopiko. Jelly candies, such as gumdrops, jujubes and gummies, use stabilizers including starch, pectin, or gelatin. Simple sugar or sucrose is turned into candy by dissolving it in water, concentrating this solution through cooking and allowing the mass either to form a mutable solid or to recrystallize. Other sugars, natural sugar substitutes, and corn syrup are also used. Gummies are typically gelatin-, pectin- or carrageenan-based chewy candies that come in a variety of shapes, colors and flavors.

Further regarding confectioneries, fondant confectionery is prepared from a warm mixture of glucose syrup and sucrose, which is partially crystallized. The fineness of the crystallites results in a creamy texture. Taffy is related to hard candy that is folded many times above 50° C. This process incorporates air bubbles, reducing the density of the material and making it opaque. Toffee, in British English, can also refer to a harder substance also made from cooked sugars which resembles toffee. Fudge is a confection of milk and sugar boiled to the soft-ball stage. Caramels are derived from mixtures of sucrose, glucose syrup, and milk products. The mixture does not crystallize, thus remains tacky. Tablets refer to crumbly milk-based soft and hard candy, based on sugars cooked to the soft-ball stage, that come in several forms, such as wafers and heart shapes. Tablets are not to be confused with tableting, a method of candy production. Liquorice confections are chewier and more resilient than gum/gelatin candies, but are still designed for swallowing, and may contain extract of the liquorice root. Chocolates are bite-sized confectioneries generally made with chocolate. Marshmallow confections include Peeps® marshmallow candies, circus peanuts, fluffy puff, etc. Marzipan confectionery is an almond-based confection, doughy in consistency, served in several different ways. Although chewing gum is uniquely made to be chewed and not swallowed, it is considered a chewy candy and a confection herein. Finally, the confectioneries of the present embodiments can include one, two, or more kinds of confections, such as candy-coated chewing gum, or chocolate-covered caramels.

In the embodiments of the present invention, the body of the confection includes natural ingredients; and does not include artificial ingredients that do not otherwise exist in nature. Natural ingredients or naturally occurring ingredients refer to any of a number of naturally occurring substances, extracts from naturally occurring substances, or highly purified components prepared from naturally occurring sources. For example, Rebaudioside A is a purified sweet steviol glycoside mixture prepared from sweet glycoside extracts obtained from Stevia rebaudiana Bertoni leaves. In some instances, however, a particular ingredient is identical regardless of its source and is considered natural. For example, natural and synthetic L-ascorbic acid are chemically identical, and there are no known differences in their biological activity. This is to be distinguished, however, from artificial ingredients consisting of synthetic compounds derived solely through chemical synthesis that have no equivalent in nature, such as, for example, brilliant blue FCF (Blue 1), a synthetic dye produced using aromatic hydrocarbons from petroleum.

The confections of the present embodiments have enhanced mouthfeel associated with the functional ingredient. Mouthfeel is a product's physical and chemical interaction in the mouth; in this case the tactile sensation a confection gives to the mouth. Mouthfeel includes such qualities as Cohesiveness: Degree to which the confection deforms before rupturing when biting with molars; Density: Compactness of cross section of the confection after biting completely through with the molars; Dryness: Degree to which the confection feels dry in the mouth; Fracturability: Force with which the confection crumbles, cracks or shatters. Fracturability encompasses crumbliness, crispiness, crunchiness and brittleness; Graininess: Degree to which a confection contains small grainy particles; Gumminess: Energy required to disintegrate a semi-solid confection to a state ready for swallowing; Hardness: Force required to deform the confection to given distance, i.e., force to compress between molars, bite through with incisors, compress between tongue and palate; Heaviness: Weight of confection perceived when first placed on tongue; Moisture absorption: Amount of saliva absorbed by confection; Moisture release: Amount of wetness/juiciness released from the confection; Mouthcoating: Type and degree of coating in the mouth after mastication (for example, fat/oil); Roughness: Degree of abrasiveness of confection's surface perceived by the tongue; Slipperiness: Degree to which the confection slides over the tongue; Smoothness: Absence of any particles, lumps, bumps, etc., in the confection; Uniformity: Degree to which the confection is even throughout; homogeneity; Uniformity of Bite: Evenness of force through bite; Uniformity of Chew: Degree to which the chewing characteristics of the confection are even throughout mastication; Viscosity: Force required to draw a liquid from a spoon over the tongue; Wetness: Amount of moisture perceived on confection's surface.

Mouthfeel can often be designed to serve a particular purpose. Thus, for example, a bite into a sour confectionery arouses a sense of play as the mouth tingles and salivates. A bite into a crunchy food product arouses a sense of play as it allows for extended mastication and the use of the muscles around the mouth. Whereas foods involved in meals are consumed to sustain biological functions and are often structurally and temporally differentiated from foods eaten as snacks, snacking is optional and simply makes the consumer feel good. Similarly, medicines are taken pursuant to package instructions or prescriptions, and although some medicines are flavored or formulated as fast-melts for children, these are not snacks that are optional and serve the purpose of providing a pleasurable experience. A snack, in contrast to a meal, is an unstructured food event in which one or more self-constrained food items may be served. Snacks, particularly confectioneries, are distinguished from meal (a scheduled and structured event) and taking medicine and typical vitamins (a scheduled and structured event, e.g., taking prescription with meal). The confections of the present invention have mouthfeel designed to appeal to the consumer's sense of playfulness and fun, providing an overall enjoyment of the confection. In particular, the confections comprise an encapsulation, for example encapsulating a functional ingredient, that snaps or crunches upon bite-through.

Furthermore, because many confections are consumed optionally and pleasurably, consumers often consider them a “guilty pleasure” because they lack nutritional value and are otherwise “empty calories.” Hence, the present embodiments provide for a confection that is not “empty calories,” so that the consumer feels less guilt about enjoying the snack. Moreover, because the nutritional aspects of the present confections are associated with a particular, pleasing mouthfeel, the consumer's knowledge of the nutritional benefit is tied to the stimulation upon eating the confection that highlight the playful dimension of the confection, providing, synergistically, a more pleasurable snacking experience.

In some embodiments, encapsulation technologies can be employed to provide a single source vehicle of enhanced nutritional benefit and enhanced mouthfeel of the present confections. In particular, the functional ingredient (i.e., nutritionally beneficial agent) is physically encased in an edible, chewable particle that is incorporated into the confection. During mastication, the functional ingredient is released from the particle. The nutritionally beneficial agents intended for use in the confectionery according to the present embodiments include the medicaments discussed herein (e.g., vitamins, anti-oxidants, essential fatty acids, electrolytes), and the vehicle can also include other minerals, vitamins, amino acids, herbs, spices, and the like. The vehicle includes at least nutritional agent and can also include natural flavorants, acidulants, tingling components, herbs, spices, colors, sweeteners and/or effervescent agents. In particular, oil-soluble spices can be compatible with water-insoluble medicaments, for example oil mints such as peppermint, spearmint and the line, and essential oils extracted from oranges, lemons and other fruits, or bean-derived flavors such as coffee, cocoa, and vanilla.

Thus, any of the confections that contain the encapsulated functional ingredient may be first identified by a visual indicator and then secondly during consumption as the encapsulation breaks open when masticated: with a palpable and, optionally, audible, bite-through. A color indicator and an audible bite-through are particularly advantageous when the confection comprises a pharmaceutical. For example, the functional ingredient is identified by color, and an audible crunch or snap also signals the presence of the medicament in the confection. This allows for the medicament to be added to a confection and preserve its integrity while also providing a visual and audible signal before and during consumption, respectively. Indeed, the presence of the dual visual and audible indicators may help from accidental ingestion of the medicament. This provides for an additional safeguard in that children will be aware that they are not consuming just a snack confection, but a food stuff that contains something they should not be consuming.

It should be noted that the functional ingredient of the present invention is not an agent for which eating typical amounts of candy snacks or chewing gum causes an overdose of an otherwise accepted level of such ingredient. In other words, the confectionery of the present invention is a snack; and is not a one-a-day-vitamin, one-a-day supplement, or a confectionery device used to deliver a particular dose of a pharmaceutical. Although the term medicament may appear and be used interchangeably with functional ingredient herein, this is for convenience only and does not imply the inclusion of pharmaceuticals in the present embodiments. Thus, for example, a serving size of the confection (e.g., one piece of gum or a single-serving package of at least a dozen candies) can include (percentage daily values based on a 2,000 calorie diet) about 2% to about 35% of at least one vitamin, such as vitamin E and/or vitamin C. The percent daily value for appropriate nutrients is a guideline established and published by the U.S. Food and Drug Administration. The percent daily value for vitamins and minerals gives a general idea of how much of a vitamin or mineral a serving contributes to the total daily requirement. For example, if the percent daily value for vitamin C of all the foods one consumes in a day adds up to 100%, that diet meets the recommendation for vitamin C.

The functional ingredients of the present embodiments are nutritive substances that are included in confections in controlled quantities in order to fulfill a specific physiological function or promote the health and well-being of the consumer. The functional ingredients of the confections can be selected from vitamins, antioxidants, electrolytes, essential fatty acids, bioflavonoids, botanicals, dental care ingredients, appetite suppressor ingredients, mouth moistening ingredients, micronutrients, throat care ingredients, nutraceuticals, nutritional supplements, traditional Chinese medicaments, homeopathic remedies, phytochemicals, and/or other bioeffecting agents. Thus, the term “functional ingredient,” as used herein, includes substances that provide some degree of nutritional or therapeutic benefit, beyond those associated with simple caloric intake, to a human when consumed. In particular, the functional ingredients may include ingredients having active effects in performance nutrition.

For example, functional ingredients comprising vitamins can include virtually any vitamin or mineral. For example, vitamin A, vitamnin C, vitamin D, vitamin E, vitamin K, vitamin B₆, vitamin B₁₂, thiamine, riboflavin, biotiri, folic acid, niacin, pantothenic acid, sodium, potassium, calcium, magnesium, phosphorus, sulfur, chlorine, iron, copper, iodine, zinc, selenium, manganese, choline, chromium, molybdenum, fluorine, and/or cobalt.

In some embodiments, vitamins may include fat-soluble vitamins such as vitamin A, vitamin D, vitamin E, and/or vitamin K. In some embodiments, vitamins may include water-soluble vitamins such as vitamin C and/or the B vitamins (thiamine or B₁, riboflavoin or B₂, niacin or B₃, pyridoxine or B₆, folic acid or B₉, cyanocobalimin or B₁₂, pantothenic acid, and/or biotin).

Further regarding vitamin C, ascorbyl palmitate is a fat-soluble version that occurs naturally in, and can be extracted from, amla fruit, the fruit of Emblica officinalis (Gooseberry tree). Once eaten, the ascorbic acid is released during digestion, and becomes bioavailable. The ascorbic acid released by the hydrolysis of ascorbyl palmitate appears to be as bioavailable as ascorbic acid alone. DeRitter, Physiologic availability of dehydro-L-ascorbic acid & palmitoyl-L-ascorbic acid, 113 Sci. 628 (1951).

Bioflavonoids or flavonoids are polyphenolic compounds found in plants, and may also be included as functional ingredients. Vitamin C-rich fruits and vegetables, especially citrus fruits, are often rich sources of flavonoids as well. The effect of bioflavonoids on the bioavailability of ascorbic acid has been examined in two published studies. A study found that a 500 mg supplement of ascorbic acid, given in a natural citrus extract containing bioflavonoids, proteins, and carbohydrates, was more slowly absorbed and 35% more bioavailable than synthetic ascorbic acid alone. Vinson & Bose, Comparative bioavailability to humans of ascorbic acid alone or in a citrus extract, 48 Am. J. Clin. Nutr. 501 (1988).

The functional ingredients of the present invention may be selected from micronutrients, which include materials that have an impact on the nutritional well-being of an organism even though the quantity required by the organism to have the desired effect is small relative to macronutrients such as protein, carbohydrate, and fat. Micronutrients may include, but are not limited to vitamins, minerals, enzymes, phytochemicals, antioxidants, and combinations thereof.

Minerals that may be included in the functional ingredients herein include sodium, magnesium, chromium, iodine, iron, manganese, calcium, copper, fluoride, potassium, phosphorous, molybdenum, selenium, and/or zinc. The inclusion of such minerals provide functional relevance as electrolytes.

Micronutrients include, for example, L-carnitine, choline, coenzyme Q10, alpha-lipoic acid, omega-3-fatty acids, omega-6-fatty acids, pepsin, phytase, trypsin, lipases, proteases, and/or cellulases.

The term “nutritional supplement” as used herein refers to a substance that exerts a physiological effect on an animal. Typically, nutritional supplements fulfil a specific physiological function or promote the health and well-being of the consumer.

Antioxidants may include materials that scavenge free radicals. In some embodiments, antioxidants may include ascorbic acid, citric acid, rosemary oil, vitamin A, vitamin E, vitamin E phosphate, tocopherols, di-alpha-tocopheryl phosphate, tocotrienois, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, co-enzym Q10, honey, and combinations thereof. Antioxidants may be found in significant amounts in plant extracts, such as Ginko Biloba leaves which contains Ginko flavonoids, Blueberry fruits which contains anthocyanids, Ginseng roots which contains gingsengnoids, and/or Eleuterococco roots which contains eleuterosids.

Phytochemicals may include but are not limited to cartotenoids, chlorophyll, chlorophyllin, fiber, flavanoids, anthocyaninis, cyaniding, delphinidin, malvidin, pelargonidin, phonolic acid, peonidin, petunidin, flavanols, catechin, epicatechin, epigallocatechin, epigallocatechingallate (EGCG), theaflavins, thearubigins, proanthocyanins, flavonols, qauercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, and/or soy isoflavonies.

The confection can also include additional ingredients to deliver heart-healthy benefits, such as Omega 3 fatty acids, flax seed oil, hemp oil, and/or chia oil.

The confection can also contain additional ingredients to deliver a diet product inducing satiety or appetite suppression, such as slimaluma, carraluma fimbriata, bauhinia extract, una de vaca, protein, nopal cactus extract, and/or prickly pear cactus extract.

The confection can include additional ingredients to deliver a natural energy blend, such as taurine, caffeine, guarana, ginseng, cordyceps, maca, reishi, maitake, Vitamin B12, and/or Vitamin B6. The confection can also include additional ingredients to deliver antioxidants, such as Vitamin E, acai, gogi, noni, blueberry, chia, and hemp oil.

The confection can also include additional ingredients to deliver an immune support blend, such as Vitamin A, Vitamin B12, Vitamin B6, Vitamin C, zinc, honey, and/or lemon. The confection can also include additional ingredients to deliver added electrolytes, such as potassium, sodium, calcium, and/or magnesium.

The terms “botanical extract” and “botanical,” as used interchangeably herein, refer to a substance derived from a plant source. Non-limiting examples may include echinacea, Siberian ginseng, ginko biloba, kola nut, goldenseal, golo kola, schizandra, elderberry, St. Johns Wort, valerian and/or ephedra.

Various herbals may also be used as functional ingredients herein, such as those with various medicinal or dietary supplement properties. Herbals are generally aromatic plants or plant parts and or extracts thereof that may be used medicinally or for flavoring. Suitable herbals may be used singly or in various mixtures. Commonly used herbs include Echinacea, Goldenseal, Calendula, Rosemary, Thymne, Kava Kava, Aloe, Blood Root, Grapefruit Seed Extract, Black Cohlosh, Ginseng, Guarana, Cranberry, Gingko Biloba, St. John's Wort, Evening Primrose Oil, Yohimbe Bark, Green Tea, Ma Huang, Maca, Bilberry, and/or Lutein.

Mouth moisteners may include, but are not limited to, saliva stimulators such as acids and salts and combinations thereof. In some embodiments, acids may include acetic acid, adipic acid, ascorbic acid, butyric acid, citric acid, formic acid, fumaric acid, glyconic acid, lactic acid, phosphoric acid, malic acid, oxalic acid, succinic acid, and/or. Mouth moisteners may also include hydrocolloid materials that hydrate and may adhere to oral surface to provide a sensation of mouth moistening. Hydrocolloid materials may include naturally occurring materials such as plant exudates, seed gums, and seaweed extracts or natural gum derivatives. In some embodiments, hydrocolloid materials may include pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, and/or bacterial gums.

Similarly, humectants which may provide a perception of mouth hydration may be included. Such humectants may include, but are not limited to glycerol, galactose, sorbitol, polyethylene glycol, erythritol, and xylitol. Additionally, in some embodiments, fats may provide a perception of mouth moistening. Such fats may include medium chain triglycerides, vegetable oils, fish oils, mineral oils, and combinations thereof.

Throat soothing functional ingredients may include natural anesthetics, demulcents, antiseptic, and combinations thereof, such as menthol, slippery elm bark, pectin, gelatin, licorice, honey, propolis, aloe vera, and combinations thereof may be included.

In some embodiments, functional ingredients that provide a perception of nasal clearing may include menthol, camphor, borneol, eucalyptus oil, peppermint oil, lavender oil, wasabi extracts, horseradish extracts, odoriferous essential oils, extracts from woods, flowers and other botanicals, and/or resins.

In other embodiments, the medicament is a homeopathic remedy, such as an herb or spice associated with homeopathic treatment. The term homeopathy comes from the Greek words homeo, meaning similar, and pathos, meaning suffering or disease. Homeopathy seeks to stimulate the body's ability to heal itself by giving very small doses of highly diluted substances. Homeopathic remedies are prepared according to the guidelines of the Homeopathic Pharmacopeia of the United States (HPUS), which was written into law in the Federal Food, Drug, and Cosmetic Act in 1938.

In yet other embodiments, the medicament is a traditional Chinese medicine such as those described in U.S. Patent Pub. No. 2010/0104518.

In particular embodiments, the confection includes at least one functional ingredient comprising a water-insoluble or lipophilic dietary supplement in an encapsulated medium with a standardized shell hardness and optional natural colorant. Example water insoluble dietary supplement medicaments include fat-soluble vitamins, essential fatty acids and antioxidants, such as vitamin E and other tocopherols and tocotrienols, vitamin D (such as ergocalciferol (vitamin D2) and choleclciferol (vitamin D3)), vitamin B2 (only slightly soluble in water), ascorbyl palmitate (a fat-soluble form of vitamin C), coenzyme Q10, L-cystine, mecobalamin (only slightly soluble in water), lipids, astaxanthin, and water-insoluble fiber. The functional ingredient of the present invention is not an agent for which eating typical amounts of candy snacks or chewing gum causes an overdose of an otherwise accepted level of such medicament. Thus, for example, a serving size of the confection (e.g., one piece of gum or a single-serving package of many candies) can include (percentage daily values based on a 2,000 calorie diet) about 2% to about 35% of at least one vitamin, such as vitamin E or vitamin C.

In some embodiments, one or more ingredients may be encapsulated with an encapsulating material. In some embodiments, partially or completely encapsulating an ingredient used in an confectionery composition with an encapsulating material may delay release of the ingredient during consumption of the confectionery composition, thereby delaying when the ingredient becomes available inside the consumer's mouth, throat, and/or stomach, available to react or mix with another ingredient, and/or available to provide some sensory experience and/or functional or therapeutic benefit. This may be particularly true when the ingredient is water soluble or at least partially water soluble. In particular, the encapsulating material of the present invention does not melt quickly in saliva, but instead provides a hard bite-through and a snap or crunch mouthfeel.

Encapsulating material for encapsulating the encapsulated functional ingredient includes any one or more natural water-soluble or water-insoluble polymers, co-polymers, or other materials capable of forming a coating, shell, or film as a protective barrier or layer around one or more functional ingredients and/or capable of forming a matrix with the one or more functional ingredients. In some embodiments, the encapsulating material may completely surround, coat, cover, or enclose a functional ingredient. In other embodiments, the encapsulating material may only partially surround, coat, cover, or enclose a functional ingredient.

Sweeteners suitable for inclusion in the functional ingredient vehicle include natural sucrose, dextrose, maltose, dextrin, xylose, ribose, glucose, mannose, galactose, fructose, invert sugar, fructo oligo saccharide, partially hydrolyzed starch, cane sugar solids, corn syrup solids, sorbitol, xylitol, mannitol, galactitol, maltitol, hydrogenated isomaltulose, isomalt, lactitol, erythritol, hydrogenated starch hydrolysate, or combinations thereof. Organic sources of sweeteners offer enhanced value to the health-conscious consumer.

In some embodiments, partially or completely encapsulating an ingredient used in a confectionery composition with an encapsulating material may stabilize the ingredient against moisture absorption and/or moisture migration.

As mentioned above, the functional ingredient vehicles and confections can also comprise other ingredients, in particular, flavors, spices, or herbs. A natural flavor or flavoring composition is a flavoring ingredient or a mixture of flavoring ingredients, solvents or adjuvants of current use for the preparation of a flavoring formulation, i.e., a particular mixture of ingredients which is intended to be added to an edible composition or chewable product to impart, improve or modify its organoleptic properties, in particular its flavor and/or taste. Flavoring ingredients are well known to a person skilled in the art, the skilled flavorist being able to select them on the basis of his general knowledge and according to the intended use or application and the organoleptic effect it is desired to achieve. See, e.g., U.S. Patent Patent Pub. No. 2009/0155445 (Le et al. Tingling & Salivating Compositions, Jun. 18, 2009); Fenaroli's Handbook of Flavour Ingredients (CRC Press, 1975). Solvents and adjuvants of current use for the preparation of a flavoring formulation are also well known in the art. They allow flavoring formulations to meet technical requirements, such as stability or tonality persistence. The solvent is most of the time part of a flavoring composition. Solvents currently used in this context include, for instance, vegetable oils or ethanol. The adjuvants, on the other hand, can have many various functions in a flavoring composition, such as stabilizers. Spices include tropic plants with aromatic fruits or barks. Herbs include plants of temperate climate featuring aromatic leaves. The range of products types and product formulations that are flavored is extensive and subjected to frequent changes, but a skilled person in the art is capable of choosing these natural ingredients as a function of the product to be flavored and of the nature of the flavoring ingredients contained in the formulation.

In some embodiments, the encapsulating material may include pectins, fats, waxes, gelatins, hydrocolloids, or oils. In some embodiments, the encapsulating material may be water-soluble or water-miscible. In such embodiments, the encapsulating material may include, but is not limited to, hydrocolloids such as starch, gum arabic, maltodextrin, and/or dextrins, and the like. In some embodiments, the encapsulating material may have a melting point from about 45° C. to about 70° C. In still other embodiments, the encapsulating material may have a melting point from about 50° C. to about 65° C.

In some embodiments, a functional ingredient may be pre-treated prior to encapsulation with an encapsulating material. For example, a functional ingredient may be coated with a “coating material” that is not miscible with the ingredient or is at least less miscible with the ingredient relative to the ingredient's miscibility with the encapsulating material.

In some embodiments, the same or different encapsulating material may be used to individually encapsulate different functional ingredients for inclusion in the same confectionery body composition. In some embodiments, the same or different methods of encapsulation may be used to individually encapsulate different functional ingredients in the same confectionery composition. For example, ascorbic acid may be encapsulated in hydrogenated soy bean oil using a spray chilling method while citric or lactic acid may be encapsulated in gum arabic using a spray driving method. Both encapsulations may then be used in the same confectionery or chewing gum composition.

The encapsulating material may incorporate natural sweeteners, flavors, starches, sensates, potentiators, breath freshening ingredients, effervescing system ingredients, lubricants, coloring agents, food acid ingredients, and combinations thereof.

In some embodiments, the encapsulant includes or is covered by a coating. The coating may also include a natural saccharide or polyol or a combination of saccharide and polyol. Suitable saccharides may include, but are not limited to, mono-saccharides, di-saccharides and poly-saccharides such as but not limited to, sucrose (sugar), dextrose, maltose, dextrin, xylose, ribose, glucose, mannose, galactose, sucromalt, fructose (levulose), invert sugar, corn syrups, cane syrups, maltodextrins, fructo oligo saccharide syrups, partially hydrolyzed starch, corn syrup solids, polydextrose, soluble fibers, insoluble fibers, and mixtures thereof. Suitable polyols may include, but are not limited to natural sugar alcohols (or polyols) such as, but not limited to, sorbitol, xylitol, mannitol, galactitol, maltitol, hydrogenated isomaltulose, lactitol, erythritol, hydrogenated starch hydrolysates, maltitol syrups, and mixtures thereof.

Sour confections or sour encapsulations typically include food acid blends, such as lactic acid, which may also be present as calcium lactate, tartaric acid, fumaric acid, and citric acid. Each of the acid components may be present in any amount to provide the desired taste. For example, the sour taste perception of an aqueous solution of 0.2% w/w of citric acid has been characterized as providing clean and refreshing tartness. See, e.g., U.S. Patent Pub. No. 2007/0269577.

In some embodiments, the functional ingredient vehicle (i.e., an encapsulation) may be sized to a particular size for use as an ingredient in a confectionery composition. For example, in some embodiments, a functional ingredient vehicle may have a particle size of 710, 420, 300, 250, 200, 100, 60, 50, 25 or 20 microns, inclusive. In some embodiments, the encapsulation may have an average particle size such as, for example. 710, 420, 300, 250, 200, 100, 60, 50, 25 or 20 microns, inclusive. In some embodiments, the encapsulation have a maximum particle size such as, for example, 710, 420, 300, 250, 200, 100, 60, 50, 25 or 20 microns, inclusive. The ultimate particle size will depend on the characteristics of the encapsulation and/or the confectionery composition and as such, other sizes are possible in other embodiments. For example, encapsulations and/or confectionery compositions with smooth, creamy textures require smaller particles sizes (below 25 microns) while in other examples, encapsulations and/or confectionery compositions with rough textures require larger particle sizes (above 250 microns). Also, in some embodiments, particles below a certain size (e.g., 25 microns) may be removed. In some embodiments, the particle size distribution may have a narrow range resulting in a sharp distribution. In some embodiments, the particle size distribution may have a wide range resulting in a smooth distribution.

A number of techniques are available for adaptation to provide for the functional ingredient vehicle(s) of the present invention, such as encapsulation, partial encapsulation or partial coating, entrapment or absorption with high or low water soluble materials or water insoluble materials. There are many ways to encapsulate one or more ingredients with an encapsulating material, including, for example, by sigma blade or Banbury™ mixer, extruder or other type of continuous mixer, spray coating, spray chilling, absorption, adsorption, inclusion complexing (e.g., creating a flavor/cyclodextrin complex, forming a glassy matrix, etc.), coacervation, fluidized bed coating, melt spinning, or other process may be used to encapsulate an ingredient with an encapsulating material. See, e.g., U.S. Patent Pub. No. 2007/0269577. The functional ingredient also may be absorbed onto an inert or water-insoluble material. Functional ingredients may be prepared in the vehicle by a multiple step process comprising any of the processes, or a combination of the processes noted. Prior to encapsulation, medicaments may also be combined with bulk sweeteners including sucrose, dextrose, fructose, maltodextrin or other bulk sweeteners, as well as sugar alcohols such as sorbitol, mannitol, xylitol, maltitol, lactitol, hydrogenated isomaltulose and hydrogenated starch hydrolyzates.

It should be noted that the encapsulation vehicles and techniques for their manufacture may be adapted to address the stability and bioavailability of particular functional ingredients. For example, the thermosensitivity of vitamins may vary in a wide range. For instance, vitamin B1 is highly thermosensitive whereas vitamin B3 (niacin) can resist very high temperatures without damage. Vitamins A, B1, B6 and E are oxygen-sensitive, and therefore should handled accordingly during manufacture.

As noted, the functional ingredient vehicle can be a particle or granule. Encapsulating compounds include, but are not limited to, sugars, gelatins, starches, cellulose, proteins, gums, sucrose esters, waxes, and polyvinyl esters and acids. For example, a tart-tasting particle can be made by first making a slurry including a medicament that has low water solubility, e.g., vitamin E, and mixing it with an aqueous acid solution of edible acids having high water solubilities, e.g., malic and tartaric acids. The slurry is then granulated and dried; the dried granules can then be further encapsulated. Some encapsulation methods consist in making particles with an insoluble surface (coacervation, coating with a fat), and applying a further coating.

As further examples, U.S. Pat. No. 4,105,801 refers to a confectionery comprising a core portion and a shell adheringly enveloping the core portion, whereby the shell is formed by an intimate mixture of microcrystals of xylitol with a solid fatty substance in a proportion of 0.5 to 15 parts by weight of fatty substance to each 100 parts by weight of xylitol. The fatty substance is preferably a mono-, di- or triglyceride having a melting range of between 20° C. and 60° C. U.S. Pat. No. 3,389,000 refers to protective coatings for granular nucleoside-5-phosphates, the coatings being edible fats derived from plants and animals that melt between 40° C. to 100° C. Examples of hydrogenated oils used are soybean oil, cottonseed oil, almond oil, castor oil, linseed oil, mustard oil, olive oil, grapefruit seed oil, palm oil, palm kernel oil, rapeseed oil, rice bran oil and the like and mixtures thereof. This reference discloses a process of preparing the granular product from a liquid mixture of fats and nucleoside-5-phosphates which are sprayed from a pressure nozzle and the resultant granules cooked and recovered.

U.S. Pat. No. 4,597,970 refers to a delivery system wherein a sweetener is coated with a mixture of fatty acid or wax, lecithin and monoglyceride. The outer layer components are maintained at a temperature of approximately 200° F. The resultant mixture after exiting the extruder is cooled to about 32° F. The solid mixture can then be milled or ground into a powder or granulated form. The size of the particles can be adjusted to accommodate a particular desired release rate and mouthfeel, depending on the vehicle, e.g., hard candies, soft candies, chewing gum compositions, and the like, in which it is incorporated. Similarly, U.S. Pat. No. 5,126,151 refers to an encapsulation of sweeteners or flavorants comprising fatty acids or waxes, lecithin, glyceride and an anti-foaming agent.

Encapsulation that employs glass-like sugars and/or polymeric materials, and several methods of creating glass-like states, are known. The glass transition temperature (Tg) represents the transition temperature from a rubbery liquid state to a glassy solid state; such a transition is characterized by a rapid increase in viscosity over several orders of magnitude and over a rather small temperature range. At temperatures below Tg, all molecular translation is halted and it is this process which provides such effective entrapping of the medicament(s) and prevention of other chemical events such as oxidation. In one approach, a homogeneous mixture of material (e.g., medicament(s), color, flavor) and carbohydrate matrix is prepared and then heated in such a way that the temperature of the mixture is greater than the Tg of the matrix, in order to form a molten mass. The molten mass is then extruded through a die. Following the extrusion step, the mixture is cooled or dried to increase the viscosity of the system, in order to render it sufficiently viscous to be able to be shaped to provide the desired particles. A typical example of extrusion techniques for preparing encapsulated volatile compounds is noted in U.S. Pat. No. 4,707,367, which refers to a process for preparing a solid essential oil composition, completely encapsulated within the extruded particulate solids. The process there-described comprises forming a homogeneous melted mixture of matrix components and essential oil flavor, and extruding the melt into a relatively cool liquid solvent. The cooling step induces the solidification and the solid extruded material is further dried and combined with an anticaking agent to produce a stable and relatively non-hygroscopic particulate essential oil composition in encapsulated form.

An alternative technology that can be adapted for use in the present embodiments is described in EP 202409 A2, which refers to a method for the production of stable, spherical particles of viable micro-organisms by mixing a culture concentrate with a bulking agent to form a homogeneous wet granulate, extruding the wet granulate through a die to produce filaments having a diameter of approximately the size of the desired spheres and then using a spheroniser device which has a plate that rotates at a tangential speed sufficient to cause the filaments to be shaped into discrete spherical particles, and finally drying the particles. Before the drying step, the glass transition temperature of the extruded mass is relatively low because of the large proportion of water used as solvent. The additional drying step is thus necessary to evaporate some water from the system, thus increasing the Tg to a sufficient value to provide a product capable of being stored at room temperature.

U.S. Pat. No. 6,607,771 (Benczedi et al., Process for the preparation of granules for the controlled release of volatile compounds, Aug. 19, 2003), refers to a process for shaping in a granular form a delivery system for the controlled release of a flavor or fragrance compound or composition by (a) preparing a mixture of a continuous phase carrier containing a volatile flavor or fragrance compound or composition finely divided therein and having a low water content so as to ensure that the Tg of said mixture is the Tg of the final product; (b) heating the mixture within a screw extruder to a temperature comprised between 90° C. and 130° C. to form a molten mass; and (c) extruding the molten mass through a die and chopping the molten mass directly as it exits the die, i.e., at the temperature of extrusion. As the molten mass exits the die, it is already in a plastic condition, thus sufficiently viscous to be cut. This approach allows the maker to shape the granule directly as the molten mass exits the die and to thus produce capsules or particles with a particularly uniform size distribution. The process may be adapted to functional ingredients described herein. In practice, the medicament is firstly dispersed by mechanical agitation in a homogeneous solution of a matrix or carrier material. The matrix can be any carbohydrate or carbohydrate derivative which can be readily processed through extrusion techniques to form a dry extruded solid. Particular examples of suitable materials include those selected from the group consisting of sucrose, glucose, lactose, maltose, fructose, ribose, dextrose, isomalt, sorbitol, mannitol, xylitol, lactitol, maltitol, pentatol, arabinose, pentose, xylose, galactose, trehalose, hydrogenated corn syrup, maltodextrin, agar, carrageenan, gums, polydextrose and derivatives and mixtures thereof. A commercially acceptable extruding apparatus is a Clextral BC 21 twin-screw extruder (Clextral, Inc., Tampa, Fla.), equipped with a cutterknife allowing to chop the melt at the die exit, when it is still plastic.

An alternative encapsulation device employs about 1% to 7% agar agar in combination with a carbohydrate material in the matrix. Agar agar is part of the family of hydrocolloids: long-chain high molecular weight polymers that disperse and hydrate in water to give a thickening and sometimes a gelling effect. Typically, an aqueous carbohydrate melt, the active material and an optional emulsifier are allowed to form a homogeneous emulsion, which is extruded under pressure through a die plate and then cooled to form a solid product containing the active material, dispersed as fine droplets. Water-insoluble components may be used in this approach, and is advantageous when the encapsulation is then used in an aqueous environment. The specific combination of a carbohydrate material with agar agar allows the formation of a gel barrier which prevents the granules from an immediate dissolution in water, thus providing a slow release of the encapsulated droplets. The invention advantageously provides a system showing a high percentage of retention of the active ingredient there-encapsulated after the particle rehydration, while being easy to manufacture with standard manufacturing equipment. U.S. Pat. No. 6,932,982 (McIver et al., Encapsulated flavor and/or fragrance composition, Aug. 23, 2005).

Typical conditions for encapsulation using agar agar carbohydrate include: (a) the carbohydrate material is mixed with a sufficient amount of water and heated to form a solution. Separately, the agar agar is mixed with approximately 11-times its weight in water and allowed to rehydrate. The agar agar suspension is then added to the carbohydrate solution which is then heated to remove sufficient water to form a viscous melt containing from 3% to 12% of water. The active ingredient and an optional emulsifier are then mixed with the melt under high shear to uniformly disperse the active ingredient throughout the melt. Alternatively, the agar agar suspension is added to the preformed emulsion consisting of the active ingredient dispersed as droplets in the carbohydrate matrix. This is then extruded under pressure through a die plate (step b)) and falls into a chilled solvent where the material is cooled to a glass, chopped and the surface washed of actives (step c)). The particles are then dried to remove residual solvent and reduce the moisture content to from 2% to 8%. The shape and size of the extruded solid can be adjusted as a function of the extrusion parameters. The temperature and pressure conditions under which this process is carried out can be adjusted by the skilled person without particular effort and as a function of the nature of the ingredients present in the melt and of the quality of the product which is desired to obtain, i.e., its granulometry and shape. The type and design of the equipment used are known in the art, as are their technical specificities and the choice of appropriate equipment for a desired specific shape and size of the extruded solid. Such extruded solids may be produced in many forms, e.g., powders of varied granulometry, rods, flakes, filaments, etc. Techniques such as grinding (or criogrinding), pulverizing or sieving are also known to further provide for reduction of the size of the extruded solid, namely in the case of extruded particulate solids, to reduce it to the state of fine micromized powders, if so desired. U.S. Pat. No. 6,932,982.

Another extrusion method suitable for the instant encapsulation, the so-called “dry-blend” extrusion techniques, typically require the use of higher pressures to feed the melt of the originally essentially solid material through the extruder than the methods which resort to the extrusion of substantially liquid or fluid mixtures of ingredients. A commercially acceptable extruding apparatus is a Clextral BC 21 twin-screw extruder equipped with a cutterknife allowing to chop the melt at the die exit, when it is still plastic. See U.S. Pat. No. 6,932,982.

The glassy, carbohydrate encapsulation vehicles can be fashioned for size and hardness as desired to achieve particular mouthfeel or audible signal (i.e., “snap” or crunch). The concentrations in which the extruded solids can be incorporated in such consumer products vary in a wide range of values, which are dependent on the nature of the product to be flavored or perfumed. Typical concentrations, to be taken strictly by way of example, are comprised in a range of values as wide as from a few parts-per-million up to 5% or even 10% of the weight of the encapsulated composition or finished confectionery into which they are included. The encapsulated water-insoluble medicament particles can also be combined, in the confection, with particles of encapsulated water-soluble components. See, e.g., U.S. Pat. No. 6,607,778 (Solid delivery systems for aroma ingredients, Mutka et al., Aug. 19, 2003)

An alternative hard encapsulation is made up of at least one fine envelope and a liquid core, as described in U.S. Pat. No. 7,744,922 (Mane et al., Capsule with fast content solubilization & release, Jun. 29, 2010). The core of the capsule is composed of water-insoluble (hydrophobic or partially soluble in ethanol) functional ingredients, or of water-insoluble functional ingredients formulated as an oil/water/oil emulsion. It may include one or more lipophilic solvents conventionally used in the food, pharmaceutical or cosmetic industries, such as triglycerides, mixtures of triglycerides such as vegetable oil, olive oil, sunflower oil, corn oil, ground nut oil, grapeseed oil, wheatgerm oil, mineral oils and silicone oils. The amount of lipophilic solvent in the core of a capsule is of the order of 0.01% to 90% of the weight of the capsule. The envelope of the capsule comprises at least one film-forming polymer as known to the person skilled in the art, in particular pectins, gelatins, natural gums (gum arabic, guar gum, carob gum, gellan gum, pullulan gum, etc.), carreghenans, cellulose derivatives, starch derivatives, etc. It also comprises at least one plasticizer, which may be of the glycerol, sorbitol, maltitol, or another polyol with plasticizing properties, and may include at least one acid. The use of at least one such acid in particular makes it possible to provide microbiological stability of the envelope of the capsule and to adjust its physicochemical and sensory properties during dissolving thereof (pH, solubility, etc.). The thickness of the envelope of the capsule according to the invention can range from between about 30 μm and about 100 μm, inclusive. The envelope can represents from about 8% to 30% of the weight of the capsule. This capsule is not a soft capsule within the meaning of the state of the art; it is a capsule which feels hard, and which can break when it is pressed too hard between the fingers. Its hardness is of the order of 1 kg/cm² to 5 kg/cm². Such a capsule, when it is ingested, generates an immediate perception in the mouth of the active compounds which it contains.

The encapsulated particle can be a non-porous, chewable, particle of elastomer; and during mastication of the confection, the nutritional agent is released from the elastomer particles over a prolonged period of time. See, e.g., U.S. Pat. No. 4,975,270 (Kehoe, Elastomer encased active ingredients, Dec. 4, 1990). Example water insoluble elastomers include rubber, chicle, crown gum nispero, balato, jetulong, pendare, perillo, niger, gutta, tunic, leche caspi, sorva, and gutta hank kang. The encapsulated agent(s) employed in chewable products can be liquid or solid form. Typically, a particle of encapsulated agent(s) has a particle size of about 0.20 mm to 1.20 mm.

The functional ingredient vehicle is included in any variety of confections as described herein or known in the art. The confections of the present invention comprise natural sweeteners. Sweeteners may include sugars, sugarless bulk sweeteners, or the like, high intensity sweeteners, or mixtures thereof. Bulk sweeteners generally are present in amounts of about 5% to about 99% by weight of the confectionery base composition. Suitable sugar sweeteners generally include mono-saccharides, di-saccharides and poly-saccharides such as but not limited to, sucrose (sugar), dextrose, maltose, dextrin, xylose, ribose, glucose, mannose, galactose, fructose (levulose), invert sugar, corn syrups, maltodextrins, fructo oligo saccharide syrups, partially hydrolyzed starch, corn syrup solids and mixtures thereof. Suitable sugarless bulk sweeteners include sugar alcohols (or polyols) such as, but not limited to, sorbitol, xylitol, mannitol, galactitol, maltitol, hydrogenated isomaltulose, lactitol, erythritol, hydrogenated starch hydrolysates, maltitol syrups, and mixtures thereof.

In some embodiments, high-intensity sweeteners also may be included as sweetening agents in the composition. Without being limited to particular sweeteners, representative categories and examples include water-soluble sweetening agents such as dihydrochalcones, monellin, stevia, steviosides, rebaudioside A, glycyrrhizin, dihydroflavenol, and sugar alcohols such as sorbitol, mannitol, maltitol, xylitol, and/or erythritol. The intense sweetening agents may be used in many distinct physical forms well-known in the art to provide an initial intense sensation of sweetness and/or a prolonged sensation of sweetness. Without being limited thereto, such physical forms include free forms, spray dried forms, powdered forms, beaded forms, encapsulated forms, and mixtures thereof.

Sweeteners may be used in amounts necessary to impart the desired effect associated with use of the functional ingredient (e.g., sweetness). In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

In some embodiments, flavorants may include those natural flavors known to the skilled artisan. These flavorings may be chosen from natural flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen, peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassaya oil. Also useful flavorings are natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles may be managed include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

In some embodiments, flavoring agents are used at levels that provide a perceptible sensory experience, i.e., at or above their threshold levels. In other embodiments, flavoring agents are used at levels below their threshold levels such that they do not provide an independent perceptible sensory experience. At sub-threshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water-soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

Potentiators may consist of materials that may intensity, supplement, modify or enhance the taste and/or aroma perception of an original material without introducing a characteristic taste and/or aroma perception of their own. In some embodiments, potentiators designed to intensify, supplement, modify, or enhance the perception of flavor, sweetness, tartness, umami, kokumi, saltiness and combinations thereof may be included.

In some embodiments, a tingling sensation may be provided. One such tingling sensation is provided by adding jambu oleoresin, or spilanthol to some examples. In some embodiments, alkylamides extracted from materials such as jambu or sanshool may be included.

Additionally, in some embodiments, a sensation is created due to effervescence. Such effervescence is created by combining an alkaline material with an acidic material. In some embodiments, an alkaline material may include alkali metal carbonates, alkali metal bicarbonates, alkaline earth metal carbonates, alkaline earth metal bicarbonates and mixtures thereof. In some embodiments, an acidic material may include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

Sensate components may also be referred to as “trigeminal stimulants.” Trigeminal stimulants are defined as an orally consumed product or agent that stimulates the trigeminal nerve. Menthol is an example of a cooling agent which is a trigeminal stimulant. Trigeminal stimulants may also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassaya oleoresin, cinnamic aldehyde, or eugenol. In addition to trigeminal nerve stimulants and cooling compounds, a cooling sensation may be provided by materials exhibiting a negative heat of solution including, but not limited to, dextrose and polyols such as xylitol, erythritol, isomalt, and sorbitol, and combinations thereof.

In addition to the techniques discussed above, commercial approaches can be used, including Durarome® encapsulated flavors, which are rod-shaped carbohydrate matrices that dissolve in aqueous solution (Firmenich); and Flexarome® particles of colored flavor, which can remain visible in the confection (Frimenich). Flexarome® particle technology can also be adapted to encapsulate the water-insolube medicaments for inclusion in confections and chewing gums of the present invention. Flexarome® particle technology produces a carbohydrate-based matrix, the medicament embedded inside, using a horizontal extrusion process. The encapsulated medicaments are stable at high temperature and high humidity, stability in limited exposure to oxygen both during and after processing, maintenance of volatile medicaments and flavors during processing, and allow for designed visual impact in size and color, controlled release upon mastication. Typical Flexarome® particle shapes include sphere/bead, irregular/shard/ or elongated/tea leaf-shapes can be selected, with relatively large sizes ranging from 0.4 mm to 13 mm.

Additionally, the encapsulation can include inner encapsulations. For example, in one embodiment an encapsulation of diameter greater than 5 mm contains encapsulations within it of diameters less than 2 mm.

The encapsulated functional ingredient may be present in amounts of about 0.1% to about 10%, inclusive, by weight of the total confectionery, such as .about 0.5% to about 7.5% by weight of the confection. With respect to their presence in the encapsulant, the functional ingredien can be present in amounts of approximately 10% to about 90%, by weight or, inclusive, of the complete encapsulation.

The process to include the encapsulation, without sacrificing its integrity, into the confection may be performed by anyone reasonably skilled in the art. General recommendations for the inclusion process are: adding the encapsulated medicament at a late stage in the process; adding the encapsulated medicament at a low temperature stage of the process; adding the encapsulated medicament at a low-shear stage of the process; adding the encapsulated medicament to the center formulation of the confection; adding the encapsulated medicament to the coating of the confection. Maintaining uniformity of the encapsulated vehicle within the confection and from individual confection to individual confection is also important. Formula modifications may need to be made to keep the encapsulation suspended properly while in-process. A gellan gum, carrageenan, xanthan gum, gum acacia, or other structure forming or thixotropic additive may need to be hydrated in the confection formula during process while the confection is in a flowing state or slurry. Additionally, modification of process equipment may need to be made to allow for the encapsulation to keep from clogging orifices or accumulating in depositing equipment. For example larger diameter lines may need to be used in this process.

The body of the natural ingredient confection is otherwise formulated and prepared by standard approaches for making confections such as hard candies, soft candies, and chewing gums. See, e.g., U.S. Patent Pub. No. 2010/0104518. For example, a hard candy can be made by mixing sugars or sweeteners, a bulking agent (e.g., Arabic powder) and water; bringing the mixture to a boil; cooling until the mixture is still capable of incorporating additional ingredients; adding the functional ingredient vehicle and flavoring or coloring agents; pouring the mixture into a mold; allowing the candy to cool and finishing the product. See, e.g., U.S. Pat. No. 6,365,209.

In other embodiments, the sensory indicator may not contain any or all of the medicament(s) present in the total confectionery. For example, vitamins can be incorporated into the formula of the confection, and the encapsulant including color and flavor placed in the confection to include the sensory indicators (e.g., palpable and, optionally, audible snap or crunch; color, shape) such that the sensory indicators are identified with the nutritional value of the confection. In particular embodiments, vitamin supplements are included in a confection that also includes hard, crunchy, pectin-encapsulated fruit flavor. In other particular embodiments, vitamin supplements are included in a confection that also includes hard, crunchy, gelatin-encapsulated natural vanilla flavor.

In one embodiment, the confection is a candy for which a single serving size (i.e., a single package consisting of multiple candy pieces), the entire serving size weighing from about 20 grams to about 60 grams, that comprises at least one of 5% to 40% calcium, 25% to 45% vitamin C, 25% to 45% vitamin E, 25% to 45% thiamin, 25% to 45% riboflavin, 25% to 45% niacin, 25% to 45% vitamin B6, 25% to 45% vitamin B12, 25% to 45% biotin, 25% to 45% pantothenic acid, and/or 4% to 8% phosphorus, based on percent daily values based on a 2,000 calorie/day diet.

The confection of the present invention may also include a “center-fill”, which refers to the innermost region of a center-fill gum or confectionery product. The “center-fill” does not require symmetry within a gum or confectionery product, only that the “center-fill” is within another region of the confectionery. In confections, more than one center-fill may be present. A center-fill may include solid, liquid, gas and mixtures thereof. The term “liquid” in the context of a center-fill includes fluid materials as well as semi-solid or gel materials. The center-fill can be aqueous, non-aqueous, or an emulsion.

A center-fill composition may include any conventional filling or combination of filling materials. The center-fill may be sugar or naturally sugar-free and it may contain fat or be fat-free. Additionally the center-fill may contain vegetable-based, dairy-based or fruit-based materials such as, but not limited to, fruit juices, fruit concentrates, fruit purees, dried fruit materials, and the like. The center-fill component may include one or more sweeteners such as those discussed herein.

In some embodiments, it is desirable to include hydrocolloid materials that increase the viscosity of the center-fill composition. The hydrocolloid materials envisioned herein are naturally occurring materials such as plant exudates, seed gums, and seaweed extracts or they may be chemically modified materials such as cellulose, starch, or natural gum derivatives. In some embodiments, hydrocolloid materials may include starches, flour, pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, and/or bacterial gums.

The texture of the center-fill can have the same or different from the texture of the rest of the confectionery composition. The texture of the center-fill can have the same or different from the appearance than the rest of the confectionery composition. The center-fill can also include one or more food acids as discussed herein. These food acids or blends thereof may be included in amounts from about 0.5% w/w to about 5.0% w/w of the center-fill composition.

The center-fill confectionery composition may include natural buffering agents, coloring, flavoring, sensates, and/or natural preservatives.

In some embodiments, the center-fill composition may include functional ingredients as discussed herein and such as, but not limited to, medicaments, nutrients such as vitamins and minerals and the like, nutraceuticals such as phytochemicals and the like, breath freshening agents, oral care agents, probiotic materials, prebiotic materials, taste and/or flavor potentiators, and throat care agents.

In some embodiments, the center-fill composition may be included in the coated center-filled chewing gum or confectionery composition in amounts from about 1% wt of the total composition to about 25% wt of the total composition.

In some embodiments, the present confection is a coated or shelled composition, for example a gummy candy shell composition comprising functional ingredient(s), sensory indicators, sweeteners, hydrocolloids, and food acids. Suitable examples of these materials are described herein and may be used with equal applicability here. The gummy candy shell composition includes functional ingredients such as, but not limited to, medicaments, nutrients such as vitamins and minerals and the like, nutraceuticals such as phytochemicals and the like, breath freshening agents, oral care agents, probiotic materials, prebiotic materials, taste and/or flavor potentiators, and throat care agents. As with the center-fill composition, in some embodiments, the gummy candy shell composition can also include buffering agents, sensates, coloring, flavoring, and preservatives.

For the hydrocolloid materials, in some embodiments, a desired texture is created by using hydrocolloids that form chewable gels when combined with the other ingredients in the gummy candy shell composition. For example, pectin and/or gelatin may be used together in a gummy candy shell composition. See U.S. application Ser. No. 10/977,585. This combination of hydrocolloids may create a gummy candy shell that sets up quickly and provides adequate shell strength and desirable texture. See also U.S. Patent Pub. No. 2007/0269577.

In some embodiments, the candy shell composition may contain sweeteners in amounts from about 35% w/w to about 75% w/w of the gummy candy shell composition. In some embodiments, the gummy candy shell composition may contain from about 0.01% w/w to about 15% w/w, such as from about 1% to about 8% w/w of hydrocolloids, inclusive.

In multiple component confectionery compositions, there is a natural tendency for moisture to migrate from areas of higher concentration to areas of lower concentration. This can result in multiple component confectionery compositions that lack desired textures as the textures can lose their differentiation as the moisture equilibrates. For example, if the gummy candy shell composition includes a center-fill, but has less moisture than the center-fill composition, the moisture will migrate out of the center-fill causing the center-fill to become firmer resulting in a center-fill texture that is similar to the gummy candy shell texture. In some embodiments, hygroscopic ingredients can be encapsulated to reduce their moisture pick up. Moisture migration can be managed by controlling the amount of bound water in the compositions. In some embodiments, the water activity is manipulated by varying the solids contents of the center-fill and confection body compositions. Water activity can be manipulated by selecting the materials used in the compositions based on their water binding capacities and using those materials in concentrations that alter the water activity of the compositions. In addition to manipulation of the solids contents and water activities of the composition; moisture migration can be managed by an intrinsic moisture resistant barrier layer. The intrinsic moisture resistant barrier layer can include a crystalline form of a saccharide or polyol. In some embodiments, the moisture resistant intrinsic moisture resistant barrier layer is formed when saccharides or polyols in the confectionery base crystallize at the surface of the confectionery base.

In other embodiment, the confection of the present invention is a chewing gum. Chewing gum compositions may be provided in a variety of different forms, such as, for example, slab, pellet, sticks, balls, cubes, center-fill gums, candy gum, multi-region gum, multi-layer gum, bubble gum, deposited gums and compressed gums. The chewing gum compositions also may include at least one flavor and a variety of optional additives. Chewing gum compositions may be provided in a variety of different forms, such as, for example, slab, pellet, sticks, balls, cubes, center-fill gums, candy gum, multi-region gum, multi-layer gum, bubble gum, deposited gums and compressed gums. The chewing gum compositions also may include at least one flavor and a variety of optional additives. The chewing gum composition also may include a gum base. The gum base may include any naturally derived component known in the chewing gum art. Such components may be water soluble, water-insoluble or a combination thereof. For example, the gum base may include elastomers (e.g., natural latexes such as chicle), bulking agents, waxes, elastomer solvents, emulsifiers, plasticizers, fillers and mixtures thereof. See, e.g., U.S. Pat. No. 7,416,752; No. 6,770,308; U.S. Patent Pub. No. 2003/0021830; No. 2010/0104518.

Chewing gum can be created using any conventional method known to those of ordinary skill in the art. In some embodiments, gum base is warmed or melted in a gum mixer to which bulk sweeteners and sweetener or polyol syrups are added and mixed until homogeneous. Flavors (which can include sensates dissolved or suspended therein), food acids, and any other ingredients are then added with mixing. Once the gum is mixed as desired, the gum mass is further processed into individual pieces. In some embodiments, a rope of chewing gum is extruded or formed and is then led into a tablet-forming mechanism including a pair of rotating chain die members which are endless chain mechanisms and both rotate at the same speed by a motor and gear mechanism. Each of the chain mechanisms include a plurality of open curved die groove members which mate and form die cavities in which the pieces of gum material (pellets or tablets) are formed. The gum pieces may be of other shapes as described above. The shape of the die groove members may be altered to provide any desired shape.

The gum may optionally be passed through a cooling tunnel either before entering the tablet-forming mechanism, after exiting the tablet-forming mechanism or both. Cooling of the rope prior to entering the tablet-forming mechanism may be beneficial to prevent rebound of the individual pieces and thus may provide an increase in productivity. The cooled pieces of gum material are then fed into a storage container for conditioning and further processing. At this point, the cooled pieces of gum material could also be fed directly into a coating tunnel mechanism, such as a rotating tunnel mechanism. In some embodiments, the chewing gum pieces are formed by rolling the chewing gum into sheets and cutting the pieces into various shapes such as cubes, rectangles, slabs, sticks, etc.

Whether the pieces of formed gum material are first stored, transported in a storage container, or fed directly into a coating tunnel or mechanism, the individual pieces of gum material may subsequently be subjected to a conventional sugar or sugarless coating process in order to form a hard exterior shell on the liquid-filled gum material. A variety of coating processes or mechanisms of this type are known. In some embodiments, the coating is applied in numerous thin layers of material in order to form an appropriate uniform coated and finished quality surface on the gum products. The hard coating material, which may include sugar, maltitol, sorbitol or any other polyol, including those described herein, and optionally flavoring, is sprayed onto the pellets of gum material as they pass through a coating mechanism or a coating tunnel and are tumbled and rotated therein. In addition, conditioned air is circulated or forced into the coating tunnel or mechanism in order to dry each of the successive coating layers on the formed products. In some embodiments, the coating, or outermost region, can be formed by lamination, dual or multiple extrusion, or any other process that creates an outermost region.

The coating composition may range from about 2% to about 80%, more specifically. about 5% to 15% by weight of an individual gum piece. The coating may include sugar or polyol such as maltitol as the primary component, but may also include flavors, colors, etc. as described below in the discussion of the gum region. The coating or outermost region may be crystalline or amorphous. The chewing gum can be coated using any of a variety of techniques. For example, the coating is applied using conventional soft or hard panning processes. These processes can include the sequential application of multiple layers of wet and dry materials that build up to form the coating. Encapsulated ingredients can be added in either the wet or dry materials or both. Moisture sensitive ingredients such as carbonated or gasified candy can be included in a particulate coating composition.

In some embodiments, chewing gum confections may include a center-fill region, which may be a liquid or powder or other solid, or gas, and a gum region. Some embodiments also may include an outer gum coating or shell, which typically provides a crunchiness or texture difference as compared to un-coated gum to the piece when initially chewed. The outer coating or shell may at least partially surround the gum region. See U.S. Patent Pub. No. 2007/0269577.

In one embodiment, the chewing gum confection consists of a one-piece serving size weighing about 2 grams to 3 grams, inclusive, and comprising at least one of 1% to 5% vitamin C, 1% to 5% vitamin E, 1% to 5% thiamin, 1% to 5% riboflavin, 1% to 5% niacin, 1% to 5% vitamin B6, 1% to 5% vitamin B12, 1% to 5% biotin, and/or 1% to 5% pantothenic acid, based on a percent daily values based on a 2,000 calorie diet.

The embodiments of the present invention provide for confection compositions and a method for delivering functional ingredients inside an encapsulated medium with a standardized shell hardness and colorant to be included into confections, such that nutritional value is added and associated with the sensory indicators in the confection. The encapsulated compositions can comprise both water-soluble and water-insoluble ingredients. The water-insoluble ingredients can be included in the confection where appropriate or may be complexed onto the shell of the encapsulated medicament. The encapsulated medicament may be included into any known confection type with various levels of moisture content. The confection composition is in the form of a hard candy, soft candy, chewing gum caramel, jelly bean, extruded food stuff, pressed tablet, coated or uncoated confection, or any other known confection type. The confection can include a homeopathic remedy as the medicament, or in addition to another medicament. The medicament-containing confection can be coated with a hard shell, which hard shell contains an additional indicator with color.

Yet another embodiment of the present invention provides for a process to produce a confection comprising an encapsulated medicament without compromising the integrity of the encapsulation itself, comprising the steps of: (a) adding the encapsulated medicament at a late stage in the process; (b) adding the encapsulated medicament at a low temperature stage of the process; (c) adding the encapsulated medicament at a low-shear stage of the process; (d) adding the encapsulated medicament to the center formulation of the confection; (e) adding the encapsulated medicament to the coating of the confection.

Packaging of the finished confection can be made of biodegradable or recyclable materials, and may bear standard labeling as required by jurisdiction.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

### Example 1

### Jelly Bean Confection Comprising Supplements

Centers include organic sugar, tapioca syrup, water, supplement premix, pectin, citric acid as shown in Table 1.

TABLE 1
Jelly bean centers

| Ingredient | Amount (% wt) |
|---|---|
| organic sugar | 40-45 |
| organic tapioca syrup | 30-35 |
| water | 15-20 |
| supplement premix | 3.75 |
| pectin | 1-5 |
| citric acid | 1-5 |

Tapioca syrup, also known as cassaya syrup, is a unique and naturally-produced sweetener made by converting the cassaya tuber or root into syrup using natural enzymes. Tapioca syrup provides a sweetness, body and humectancy that is similar to high fructose corn syrup but this natural sweetener is considered hypoallergenic. An example supplement premix is found in Table 5, herein.

For coating, jelly bean centers were placed into a coating pan. Tapioca syrup, malic acid and color were mixed, and added to centers in amount sufficient to wet the centers. Flavor beads were mixed in, and the color of beads was used to visually check for even distribution among the centers. Immediately upon even distribution, dried cane juice sugar was added until the coated centers were dry. This process was repeated until the jelly beans weighed between not more than 1.2 g/bean, then the jelly beans were sealed with capol 155C (ethanol), and visually inspected for quality.

The final jelly bean product contains organic sugar, tapioca syrup, evaporated cane juice, pectin, citric acid, malic acid, and less than 2% wt of natural flavors, gelatin, medium chain triglycerides, potassium phosphate, maltodextrin, calcium phosphate, ascorbic acid (vitamin C), d-α-tocopheryl acetate (vitamin E), thiamine hydrochloride (vitamin B1), riboflavin (vitamin B2), calcium-d-pantotheate (vitamin B5), pyridoxine hydrochloride (vitamin B6), cyanocobalamin (vitamin B12), biotin, and natural color.

A 28 gram serving contains 90 calories, no fats; and as a percent of daily values, based on a 2,000 calorie diet: 1% sodium, 3% potassium, 8% total carbohydrates, 35% vitamin C, 4% calcium, 35% vitamin E, 35% vitamin B6, 35% vitamin B12, 35% biotin, 35% pantothenic acid, and 6% phosphorus.

### Example 2

### “Gummy” Confection Comprising Dietary Supplement

Dry ingredients and liquids were blended in a mixing vessel according to the amounts shown in Table 2 and then cooked.

TABLE 2
Gummy centers

| Ingredient | Amount (% wt) |
|---|---|
| organic sugar | 40-45 |
| organic tapioca syrup | 30-35 |
| water | 15-20 |
| pectin | 1-5% |

After cooking, citric acid (1-5% wt), natural color (0-1% wt), flavors (0-1% wt) and supplement premix (3.75% wt, see Table 5 example supplement premix, herein) were added. The mixture was then starch-conditioned, molded, cured, oiled, and inspected before packaging.

The final product in this example contains organic tapioca syrup, organic evaporated cane juice, pectin, carnauba wax, citric acid, sunflower oil, and less than 2% wt of stearic acid, silicon dioxide (natural), gelatin, medium chain triglycerides, sorbitol, potassium phosphate, maltodextrin, calcium phosphate, ascorbic acid (vitamin C), d-α-tocopheryl acetate (vitamin E), thiamine hydrochloride (vitamin B1), riboflavin (vitamin B2), calcium-d-pantotheate (vitamin B5), pyrixidone hydrochloride (vitamin B6), cyanocobalamin (vitamin B12), niacinamide (vitamin B3), biotin, and natural color.

One serving of twelve gummy candies, 40 grams, has 80 calories, 5 calories from non-trans fat; and as a percent of daily values, based on a 2,000 calorie diet: 3% potassium, 20% fiber, 9% total carbohydrates, 35% vitamin C, 15% calcium, 35% vitamin E, 35% thiamin, 35% riboflavin, 35% niacin, 35% vitamin B6, 35% vitamin B12, 35% biotin, 35% pantothenic acid, and 6% phosphorus.

In a consumer taste test, the confection averaged 6.6 (in a scale of 1 (poor) to 7 (excellent) for flavor, taste, mouthfeel, texture, and odor. The confection averaged 5.7 for appearance. One consumer stated “It looks healthy.”

### Example 3

### Hard Candy Confection Comprising Supplements

An example confection is a “tart.” Ingredients for grape, raspberry and orange flavored tarts including dietary supplements (example supplement premix in Table 5, herein) as shown in Table 3:

TABLE 3
Tart candy

| Ingredient (% wt) | Raspberry | Grape | Orange |
|---|---|---|---|
| xylitol | 36.78 | 36.45 | 36.79 |
| sorbitol | 34.76 | 34.77 | 34.77 |
| oryza (rice dextrose) | 3.75 | 1.25 | 3.75 |
| fibersol II | 5.41 | | 5.41 |
| stearic acid | 8.90 | 8.90 | 8.94 |
| calcium carbonate | 2.16 | 2.16 | 2.16 |
| silicon dioxide | 0.81 | 0.5 | 0.81 |
| citric acid | 4.48 | 4.48 | 4.48 |
| natural grape flavor | | 3.00 | |
| raspberry flavor | 0.45 | | |
| orange oil flavor | | | 0.41 |
| supplement pre mix | 1.68 | 1.68 | 1.68 |
| vanilla hardcaps | 1.00 | 1.00 | 0.81 |
| exbery cherry red | | 0.40 | |
| exberry intense pink | 0.40 | | |
| maltodextrin | | 5.4 | |

Vanilla flavor hardcaps include gelatin, mixed trigycerides, sorbitol, silicon dioxide (anti-caking agent), and carmine (color). All ingredients, except flavoring oils, were weighed and sifted through a #12 screen, and blended for 20 min. Then, flavoring oil was added, blended, and the mixture was allowed to dry and further processed by tableting.

The final product in this example contains xylitol, sorbitol, soluble corn fiber, citric acid, natural flavor, calcium carbonate, and less than 2% wt of whole grain brown rice syrup solids, silicon dioxide (natural), gelatin, medium chain triglycerides, potassium phosphate, maltodextrin, calcium phosphate, ascorbic acid (vitamin C), d-α-tocopheryl acetate (vitamin E), thiamine hydrochloride (vitamin B1), riboflavin (vitamin B2), calcium-d-pantotheate (vitamin B5), pyridoxine hydrochloride (vitamin B6), cyanocobalamin (vitamin B12), niacinamide (vitamin B3), biotin, and natural color.

One serving of 28 grams (multiple candies) has 90 calories; and as a percent of daily values, based on a 2,000 calorie diet: 3% potassium, 4% fiber, 8% total carbohydrates, 35% vitamin C, 30% calcium, 35% vitamin E, 35% thiamin, 35% riboflavin, 35% niacin, 35% vitamin B6, 35% vitamin B12, 35% biotin, 35% pantothenic acid, and 6% phosphorus.

Upon mastication, the hardcaps provide an enhanced snap or pop of texture and flavor compared with tarts that lack hardcaps. This added enhanced mouthfeel, combined with the nutritional supplements provided by the snack, improves the candy consumption experience. In a consumer taste test, the confection averaged >5 (in a scale of 1 (poor) to 7 (excellent) for appearance, flavor, taste, flavor, and texture. One consumer reported “It tastes sweet, but not too sweet;” and another stated “It gets a boost just because it's healthy.”

### Example 4

### Hard Candy Confection Comprising Supplements

An additional example “tart” confection is provided. Ingredients for grape, raspberry and orange flavored tarts, including dietary supplements (Vitamin Premix, see Table 5, herein), are shown in Table 4:

TABLE 4
Tart candy

| Ingredient | Raspberry | Grape | Orange |
|---|---|---|---|
| Xylitol | 46.22 | 43.28 | 46.36 |
| Sorbitol | 43.21 | 40.75 | 43.32 |
| Magnesium Stearate | 1.00 | 1.00 | 1.00 |
| Calcium Carbonate | 2.16 | 2.16 | 2.16 |
| Silicon Dioxide | 0.00 | 0.00 | 0.00 |
| Citric Acid | 4.48 | 4.48 | 4.48 |
| Natural Flavor (Sweet Type) | 0.50 | 0.50 | 0.50 |
| Flexarome ® SweetGem | | | |
| Natural Orange flavor | | | 0.50 |
| Natural Strawberry flavor | 0.75 | | |
| Natural Grape flavor | | 0.75 | |
| Vitamin Premix | 1.68 | 1.68 | 1.68 |
| Maltodextrin | | 5.40 | |
| TOTAL | 100.00 | 100.00 | 100.00 |

Flexarome ® technology provides flavor embedded inside a carbohydrate matrix, made using a horizontal extrusion process by Firmenich (Geneva, CH).

### Example 5

### Confection Comprising Homeopathic Remedy for Allergies

A candy confection containing honey, quercetin (Allium cepa), ascorbyl palmitate, bromelain (Ananas comosus), N-acetyl cysteine, euphrasia (Euphrasia nemorosa), and stinging nettle (Urtica dioica) is prepared for homeopathic treatment for allergies.

Quercetin, from Allium cepa, a water-insoluble substance found in the skin of onions and apples, is a natural antihistamine and particularly good for a drippy nose. Bromelain, from pineapple (Ananas comosus), is also beneficial for allergies. N-Acetyl L-Cysteine is an amino acid that helps thin mucus. Stinging nettle leaf is often included in homeopathic allergy remedies. Euphrasia is a homeopathic remedy considered especially beneficial in relieving burning, itchy eyes.

Water-insoluble medicaments quercetin dehydrate and ascorbyl palmitate are mixed with triglycerides, orange oil (flavor) and natural color, and enveloped in a film-forming natural, vegan polymer (such as carrageenan), to form small, hard capsules. See, e.g., U.S. Pat. No. 7,744,922. Alternatively, an aqueous organic sugar, agar agar melt is prepared, then the quercetin dehydrate, ascorbyl palmitate and orange oil added and mixed to form a homogeneous emulsion, and the melt is extruded under pressure through a die plate and then cooled to form a solid encapsulement. See U.S. Pat. No. 6,932,982. Flexarome® particle technology can also be adapted to encapsulate the homeopathic remedies in a carbohydrate matrix, as described in, e.g., U.S. Pat. No. 5,786,017; WO 00/25606; WO 01/17372; WO 02/065858; WO 03/056938.

The components for the body of the confection, e.g., organic sugar, honey, water and pectin, are mixed and stoved. After cooling, natural color, flavors, N-acetyl cysteine USP, stinging nettle leaf powder, euphrasia extract, and the quercetin/ascorbyl palmitate capsules are added. The confection is processed, inspected, and packaged.

A serving of this confection (multiple candies) will include 250 mg quercetin, 200 mg stinging nettle leaf powder, 50 mg bromelian (2400 GDU), 25 mg N-acetyl cysteine, and 470 mg euphrasia.

### Example 6

### Chewing Gum Comprising Supplements

The supplement premix is described in Table 5:

TABLE 5
Supplement premix

| Ingredient | Market Form | USRDI % | Per 1.41 g | |
|---|---|---|---|---|
| Vitamin C | Ascorbic Acid | 100 | 60 | mg |
| Vitamin E | d-α Tocopheryl Acetate | 100 | 30 | IU |
| Vitamin B1 | Thiamine Hydrochloride | 100 | 1.5 | mg |
| Vitamin B2 | Riboflavin | 100 | 1.7 | mg |
| Pantothenic Acid | Calcium D-Pantothenate | 100 | 10 | mg |
| Vitamin B6 | Pyridoxine Hydrochloride | 100 | 2 | mg |
| Vitamin B12 | Cyanocobalamin | 100 | 6 | μg |
| Vitamin B3 | Niacinamide | 100 | 20 | mg |
| Biotin | Biotin | 100 | 300 | μg |
| Calcium | Calcium Phosphate | 10 | 100 | mg |
| Potassium | Potassium Phosphate | 10 | 350 | mg |
| Phosphorus | Calcium Phosphate, | 19.02 | 190.22 | mg |
| | Potassium Phosphate | | | |
| Q.S. | Maltodextrin | | | |

The ingredients of a snap citrus sugar free chewing gum are shown in Table 6:

TABLE 6
“SNAP” citrus sugar free chew

| Ingredient | % wt |
|---|---|
| Manga T | 35.00 |
| Triacetin | 0.15 |
| Sorbitol | 10.35 |
| Xylitol-CM90 | 43.85 |
| Xylitol-C | |
| Stevia extract | 0.08 |
| Mag 100 | 0.15 |
| Natural lemon | 0.29 |
| Natural lime | 0.29 |
| Natural orange 5-fold | 0.864 |
| Citric acid | 0.825 |
| Malic acid | 0.275 |
| Encapsulated citric acid | 1.3750 |
| Flavor beads | 0.500 |
| Titanium oxide | 1.00 |
| TOTAL | 100.00 |

Gum bases and triacetin were added to a warm mixer and the blades started. Xylitol-C and titanium oxide were added and mixed until the base was soft and ingredients incorporated. Then, ½ of the xylitol was added slowly until mixed, and then the remaining xylitol, encapsulated citric acid, citric acid, and malic acid were added and mixed for about 5 min. Stevia extract, Mag 100 and supplement premix were added, and mixing continued for about 2 min. Liquid flavors were added and mixed 3 min to 5 min until the batch was uniform. Flavor beads were added and mixed 1 min to 2 min. The gum was then mill rolled with a target weight of 1.68 gm/piece. In traying, gum centers weighed 35 lbs per tray. Coating was done at 6 trays/pan.

The final product in this example contains xylitol, mannitol, gum base, and less than 2% wt of gum Arabic, titanium dioxide (colorant), citric acid, glycerin, soy lecithin, malic acid, resinous glaze, canauba wax, beta carotene, gelatin, medium chain triglycerides, sorbitol, potassium phosphate, maltodextrin, calcium phosphate, ascorbic acid (vitamin C), d-α-tocopheryl acetate (vitamin E), thiamine hydrochloride (vitamin B1), riboflavin (vitamin B2), calcium-d-pantotheate (vitamin B5), pyridoxine hydrochloride (vitamin B6), cyanocobalamin (vitamin B12), niacinamide (vitamin B3), biotin, and natural color.

A serving size of one piece of this confection (2.5 grams) contains 5 calories; and as a percent of daily values, based on a 2,000 calorie diet: 1% total carbohydrates, 2% vitamin C, 2% vitamin E, 2% thiamin, 2% riboflavin, 2% niacin, 2% vitamin B6, 2% vitamin B12, 2% biotin, and 2% pantothenic acid.

Consumers report pleasing flavors that change as the gum is chewed. Consumers reported a noticeable and pleasing mouthfeel associated with the palpable snap of the hardcap beads. In a consumer taste test, the confection averaged >5 (on a scale of 1 (poor) to 7 (excellent)) for flavor, taste, mouthfeel. The confection averaged >6 for color, odor, shape, color, and texture. One user stated “The gum fits my healthy eating criteria.”

### Example 7

### Caramel Confection Comprising Supplements

Ingredients for a “MeI” candy are shown in Table 6 (example vitamin premix shown in Table 5, herein). Briefly, cane juice was dissolved in water and brought to a boil. Tapioca, sweetened condensed milk, palm kernel oil, lecithin, salt were added, and the mixture cooked to 242° C., then cooking process stopped. Natural vanilla flavor, the vitamin blend, and vanilla hardcap beads were blended and the mixture transferred to a mogul machine (available, e.g., from Bosch GmbH), molded to 1.25 g per impression, cured at ambient temperature for at least 24 hr until centers were firm enough for panning Starch was removed via shakeout and the candies removed from the Mogul machine and chocolate coated. The chocolate-coated caramels were then air-polished, inspected, and packaged.

TABLE 6
MEL candy ingredients (wt %)

| | |
|---|---|
| Water | 5.90 |
| Tapioca Syrup | 41.00 |
| Evaporated Cane Juice | 16.00 |
| Sweetened Condensed Milk | 20.90 |
| Palm Kernel Oil | 12.00 |
| Unbleached Soy Lecithin | 0.17 |
| Salt | 0.97 |
| Vanilla | 0.48 |
| Vitamin Premix | 1.78 |
| Hardcap beads | 0.80 |

The confection produced by this process contains tapioca syrup, sweetened condensed milk (milk, skim milk, sugar), milk chocolate (sugar, whole milk, cocoa butter, chocolate liquor, soy lecithin (emulsifier), vanilla), evaporated cane juice, fractionated palm kernel oil, water, and 2% or less of natural flavoring, salt, soy lecithin, gelatin, medium chain triglycerides, potassium phosphate, maltodextrin, calcium phosphate, ascorbic acid (vitamin C), d-α-tocopheryl acetate (vitamin E), thiamine hydrochloride (vitamin B1), riboflavin (vitamin B2), calcium-d-pantotheate (vitamin B5), pyridoxine hydrochloride (vitamin B6), cyanocobalamin (vitamin B12), niacinamide (vitamin B3), and biotin.

A single 41 g serving (multiple candies) contains 160 calories, 60 from fats; and as a percent of daily values, based on a 2,000 calorie diet: 11% total fats, 18% saturated fats, 6% sodium, 3% potassium, 8% total carbohydrates, 1% protein, 35% vitamin C, 9% calcium, 35% vitamin E, 35% thiamin, 35% riboflavin, 35% niacin, 35% vitamin B6, 35% vitamin B12, 35% biotin, 35% pantothenic acid, and 6% phosphorus.

Consumers reported a noticeable and pleasing mouthfeel associated with the palpable snap of the hardcap beads. In a consumer taste test, the confection averaged 6.9 (in a scale of 1 (poor) to 7 (excellent) for flavor, taste and mouthfeel. The confection averaged >6.5 for color, odor, and texture.

We claim:

1. An edible snack confectionery composition comprising:
a confectionery main body comprising natural ingredients; and
a confectionery functional ingredient vehicle within the main body;
wherein the vehicle provides at least one sensory signal to the consumer, wherein the sensory indicator is a palpable snap, crunch, or pop characterized by a hard bite-through.

2. The edible snack confectionery composition of claim 1, wherein the functional ingredient provides electrolytes and is at least one of potassium, sodium, calcium, or magnesium.

3. The edible snack confectionery composition of claim 1, wherein the functional ingredient is at least one ingredient characterized by heart-healthy benefit, selected from Omega 3 fatty acids, flax seed oil, hemp oil, or chia oil.

4. The edible snack confectionery composition of claim 1, wherein the functional ingredient is at least one ingredient characterized as supporting the immune system, selected from Vitamin A, Vitamin B12, Vitamin B6, Vitamin C, zinc, honey, or lemon.

5. The edible snack confectionery composition of claim 1, wherein the functional ingredient is at least one ingredient characterized by inducing satiety or appetite suppression, selected from Slimaluma, carraluma fimbriata, bauhinia extract, una de vaca, protein, nopal cactus extract, or prickly pear cactus extract.

6. The edible snack confectionery composition of claim 1, wherein the functional ingredient is at least one ingredient characterized a providing natural energy, selected from taurine, caffeine, guarana, ginseng, cordyceps, maca, reishi, maitake, Vitamin B12, or Vitamin B6.

7. The edible snack confectionery composition of claim 1, wherein the functional ingredient is at least one antioxidant, selected from Vitamin E, acai, gogi, noni, blueberry, chia, or hemp oil.

8. The edible snack confectionery composition of claim 1, wherein the functional ingredient is homeopathic remedy.

9. The edible snack confectionery composition of claim 1, wherein the functional ingredient is sensitive to light, heat, water, oxidation or is incompatible with ingredients in the confection composition.

10. The edible snack confectionery composition of claim 1, wherein the functional ingredient vehicle is an encapsulation

11. The encapsulation of claim 10, wherein the functional ingredient vehicle is an encapsulation having an average diameter of from 5 μm to 800 μm, inclusive.

12. The encapsulation of claim 10, wherein the encapsulation comprises a water-insoluble functional ingredient.

13. The encapsulation of claim 10, wherein the encapsulation comprises pectin.

14. The encapsulation of claim 10, wherein the encapsulation comprises gelatin.

15. The encapsulation of claim 10, wherein the encapsulation is comprised of layers of coatings.

16. The encapsulation of claim 10, wherein the encapsulation is an encapsulation of diameter greater than 5 mm and contains encapsulations within it of diameters less than 2 mm.

17. The encapsulation of claim 10, wherein the encapsulation comprises an outer shell that includes a water-soluble functional ingredient.

18. The encapsulation of claim 10, wherein the encapsulation contains a color.

19. The encapsulation of claim 10, wherein the encapsulation provides for at least one audible sensation during mastication.

20. The encapsulation of claim 10, wherein the encapsulation has a standardized hardness.

21. The edible snack confectionery composition of claim 10, wherein the snack confectionery composition is in the form of a hard candy, soft candy, chewing gum, chocolate, caramel, jelly bean, pressed tablet, or coated or uncoated confection.

22. The composition of claim 21, wherein the confection is further coated with a hard shell comprising an additional sensory indicator with color.

23. An edible snack confectionery composition comprising:
a confectionery main body comprising natural ingredients; and
a confectionery functional ingredient vehicle within the main body;
wherein the vehicle provides at least one sensory signal to the consumer, wherein the sensory indicator is a palpable snap, crunch, or pop characterized by a hard bite-through; and
wherein a single serving size weighing from about 20 grams to about 60 grams and consisting of a plurality of pieces, provides at least one functional ingredient selected from 5% to 40% calcium, 25% to 45% vitamin C, 25% to 45% vitamin E, 25% to 45% thiamin, 25% to 45% riboflavin, 25% to 45% niacin, 25% to 45% vitamin B6, 25% to 45% vitamin B12, 25% to 45% biotin, 25% to 45% pantothenic acid, or 4% to 8% phosphorus; based on U.S. FDA percent daily values based on a 2,000 calorie/day diet.

24. An edible snack confectionery composition comprising:
a confectionery chewing gum main body comprising natural ingredients; and
a confectionery functional ingredient vehicle within the main body;
wherein the vehicle provides at least one sensory signal to the consumer, wherein the sensory indicator is a palpable snap, crunch, or pop characterized by a hard bite-through; and
wherein a single serving size of one piece of chewing gum, weighing from about 1 gram to about 2 grams, provides at least one functional ingredient selected from 1% to 5% vitamin C, 1% to 5% vitamin E, 1% to 5% thiamin, 1% to 5% riboflavin, 1% to 5% niacin, 1% to 5% vitamin B6, 1% to 5% vitamin B12, 1% to 5% biotin, or 1% to 5% pantothenic acid; based on U.S. FDA percent daily values based on a 2,000 calorie/day diet.

25. A process to include the encapsulated functional ingredient vehicle of claim 10 in an edible snack confection without compromising the integrity of the encapsulation itself comprising at least one of the following steps: (a) adding the encapsulated medicament at a late stage in the process; (b) adding the encapsulated medicament at a low temperature stage of the process; (c) adding the encapsulated medicament at a low-shear stage of the process; (d) adding the encapsulated medicament to the center formulation of the confection; (e) adding the encapsulated medicament to the coating of the confection.
